(19) United States
(12) Patent Application Publication
(10) Pub. No.: US 2011/0112344 A1
(43) Pub. Date: May 12, 2011

(54) PROCESS AND INTEGRATED SYSTEM FOR THE PREPARATION OF A LOWER OLEFIN PRODUCT

(76) Inventors: Leslie Andrew Chewter, Amsterdam (NL); Jeroen Van Westrenen, Amsterdam (NL)

(21) Appl. No.: 12/942,851

(22) Filed: Nov. 9, 2010

(30) Foreign Application Priority Data
Nov. 10, 2009 (EP) 09175611.4

(51) Int. Cl. C07C 4/02; B01J 8/00; C07C 4/06

(52) U.S. Cl. 585/302; 422/187

(57) ABSTRACT

A process for the preparation of an olefin product comprising ethylene and/or propylene, which process comprises the steps of
a) cracking a paraffin feedstock comprising C2-C5 paraffins under cracking conditions in a cracking zone to obtain a cracker effluent comprising olefins;
b) converting an oxygenate feedstock in an oxygenate-to-olefins conversion system, comprising a reaction zone in which an oxygenate feedstock is contacted with an oxygenate conversion catalyst under oxygenate conversion conditions, to obtain a conversion effluent comprising ethylene and/or propylene;
c) combining at least part of the cracker effluent and at least part of the conversion effluent to obtain a combined effluent, and separating an olefin product stream comprising ethylene and/or propylene from the combined effluent, wherein the cracker effluent and/or the conversion effluent comprises a C4 portion comprising unsaturates, and wherein the process further comprises at least partially hydrogenating at least part of the C4 portion, to obtain an at least partially hydrogenated C4 feedstock; and recycling at least part of the at least partially hydrogenated C4 feedstock as recycle feedstock to step a) and/or step b), and an integrated system for performing the process.

This application claims the benefit of European Application No. 09175611.4 filed Nov. 10, 2009, which is incorporated herein by reference.

### BACKGROUND

This invention relates to a process and integrated system for the preparation of a lower olefin product, in particular including lower olefins such as ethylene and/or propylene. More in particular this invention relates to an integrated process and system including the cracking of light paraffins such as ethane to lower olefins, and the conversion of oxygenates such as methanol and/or dimethylether into lower olefins.

In U.S. Pat. No. 6,049,017 and U.S. Pat. No. 5,914,433 processes are described for producing lower olefins, such as for example a steam cracking process. In the processes of U.S. Pat. No. 6,049,017 and U.S. Pat. No. 5,914,433, the effluent of the steam cracking process is fractionated. A C4 olefin and diolefins fraction is first selectively hydrogenated to hydrogenate the diolefins and subsequently the iso olefins in the fraction subjected to an etherfication process with methanol to MTBE ether. The remaining olefins are cracked to lower olefins and combined with the effluent of the steam cracking process. In the processes of U.S. Pat. No. 6,049,017 and U.S. Pat. No. 5,914,433 part of the C4 olefinic fraction is converted to MTBE ether.

The article “Integration of the UOP/HYDRO MTO Process into Ethylene plants” by C. N. Eng et al., 10th Annual Ethylene Producers' Conference, 1998, New Orleans, La., discloses synergies between a methanol-to-olefins (MTO) unit and a cracker. Excess low-level heat from a steam cracker can provide some of the heat for vaporization for the methanol in the UOP/HYDRO MTO unit. Also, small amounts of ethane and propane produced with the latter process can be recycled to the pyrolysis furnaces to enhance overall yields. A process flow scheme for the revamp of an existing ethylene plant by adding a UOP/HYDRO MTO unit to a cracker is disclosed in the paper “UOP/Hydro MTO Applications” by C. N. Eng et al; Asian Olefins and Derivatives Conference, Asian Chemical News/Dewitt, Singapore Jun. 18-19, 1997, as well as in a slide presentation marked UOP, and cited in the prosecution file of US patent application published as US2005/0038304 A1.

An integration scheme of an etylene plant and an MTO reactor is also disclosed in the article “MTO—An Alternative for Ethylene Production?” by H. Zimmermann, ABL Ethylene Symposium, November 1999, Orlando, Fla. Also US2005/0038304 A1 discloses an integrated system for producing ethylene and propylene from an oxygenate to olefin (OTO) reaction system and a steam cracking system, in particular a naphtha cracking system, wherein effluents from a cracking furnace and from an MTO reactor are at least partially combined.

WO2009/039948 A2 discloses a process for producing C2-C4 olefins by using an integrated system of a methanol-to-propylene (MTP) reactor and a steam cracker, so as to increase the production of propylene. In the MTP reactor a shape-selective zeolite, in particular ZSM-5, is used. Moreover, ethane and propane are recycled to the cracker. Methane/light ends, as well as a C4/C4=stream after butadiene extraction, are at least partly recycled to the MTP reactor. Butadiene is obtained as a product. A C5/C6 product stream is also recycled to the MTP reactor.

The availability of hydrocarbon feedstocks at certain locations can be a challenge for running existing plants like steam crackers, or can be insufficient to build new plants of sufficient size to achieve economy of scale. A particular issue can for example be diminishing supply of ethane originating from a successively depleting natural gas field to an ethane cracker.

It is desired to maximise the production of ethylene and/or propylene in an integrated process including steam cracking and oxygenate-to-olefins conversion.

### SUMMARY OF THE INVENTION

The present invention provides a process for the preparation of an olefin product comprising ethylene and/or propylene, which process comprises the steps of

a) cracking a paraffin feedstock comprising C2-C5 paraffins under cracking conditions in a cracking zone to obtain a cracker effluent comprising olefins;
b) converting an oxygenate feedstock in an oxygenate-to-olefins conversion system, comprising a reaction zone in which an oxygenate feedstock is contacted with an oxygenate conversion catalyst under oxygenate conversion conditions, to obtain a conversion effluent comprising ethylene and/or propylene;
c) combining at least part of the cracker effluent and at least part of the conversion effluent to obtain a combined effluent, and separating an olefin product stream comprising ethylene and/or propylene from the combined effluent,
wherein the cracker effluent and/or the conversion effluent comprises a C4 portion comprising unsaturates, and wherein the process further comprises at least partially hydrogenating at least part of the C4 portion, to obtain an at least partially hydrogenated C4 feedstock; and recycling at least part of the at least partially hydrogenated C4 feedstock as recycle feedstock to step a) and/or step b).

The C4 portion is a hydrocarbon compound having 4 carbon atoms, or a mixture including hydrocarbon compounds having 4 carbon atoms, and comprises unsaturates such as butene and/or butadiene and/or vinylacetylene. Typically the C4 portion comprises at least 5 wt % unsaturates, in particular more than 10 wt %, more in particular more than 20 wt %. Butene can for example be 1-butene, 2-butene, iso-butene, or mixtures including two or more of these compounds. The C4 portion may also comprise saturates such as butane (n-butane and/or iso-butane). In one embodiment, at least part of the C4 portion can be isolated from other components of the combined effluent, so that it forms a concentrated C4 stream comprising 50 wt % or more of species having 4 carbon atoms, in particular 75 wt % or more, more in particular 90 wt % or more.

In other embodiments, the at least part of the C4 portion forms part of a mixed stream comprising C3 and higher (C3+), or C4 and higher (C4+) hydrocarbons, such as a stream substantially containing hydrocarbons in the range of C3-C8, or C4-C7, in particular C4-C6 or C4-C5. In addition to C4 unsaturates such a mixed stream typically comprises other unsaturates as well.

By at least partially hydrogenating at least part of the C4 portion, valuable feedstock for recycling to the integrated process is obtained, generating additional ethylene and/or propylene. At least partially hydrogenating can include hydrogenating di-olefins and/or acetylenes to obtain additional butene, and recycling butene preferably to step b), and/or hydrogenating to butane, and recycling butane to a cracking step.

In one embodiment the recycle feedstock comprises butene, preferably at least 10 wt % butene, and the recycle feedstock comprising butene is recycled to step b). In particular, the recycle feedstock comprising butene can be contacted with the oxygenate conversion catalyst in the oxygenate-to-olefins reaction zone. Such an olefinic co-feed can generate additional ethylene and propylene in the course of the OTO reaction, in particular when the OTO conversion system is designed to receive such olefinic co-feed. The oxygenate-to-olefins conversion system can also comprise an olefin cracking zone separate from the oxygenate-to-olefins reaction zone, and the recycle feedstock including butene can be fed to the olefin cracking zone. The olefin cracking zone suitably comprises a catalyst. The recycle feedstock can include higher olefins such as C4-C6 olefins.

In one embodiment the C4 portion comprises butadiene, and the recycle feedstock comprises butene obtained from hydrogenating butadiene. Butadiene is typically formed much more in a cracking system than in an OTO conversion system. In an integrated system, a C4 fraction of the combined effluent comprises significant quantities of butadiene. Butadiene can cause fouling and coking when present in a recycle stream. Extraction of butadiene is possible, but costly, and total recovery is relatively small so that there is not always a commercial outlet. By selective hydrogenation of butadiene, butene is formed that can be recycled as additional feedstock for conversion to ethylene and propylene, in particular to step b).

The C4 portion typically also comprises butane, and/or butane can be obtained from hydrogenation of unsaturates in the C4 portion. A recycle feedstock comprising butane, preferably at least 10 wt % butane, such as at least 50 wt % butane, can be recycled to step a). It is found to be advantageous in this case to select the cracking conditions in the cracking zone such that 97 wt % or less of the butane is converted during a single pass through the cracking zone, which improves the distribution of cracking products from butane such that maximum ethylene and propylene can be formed in the integrated process.

In one embodiment a recycle feedstock comprising butane, preferably at least 10 wt % butane such as at least 50 wt % butane, is subjected to cracking under low severity cracking conditions, wherein butane conversion is 90 wt % or less, to obtain a butane cracking effluent. Cracking effluent from low severity butane cracking can be used fully or in part as feed to step b). In a particular embodiment the low severity butane cracking can be achieved by adding the butane-comprising recycle feedstock to steam, before or after subjecting the steam to superheating, which steam is subsequently fed to the OTO reaction zone. This superheating can be conducted in one of a superheating furnace or a superheating zone in a convection section of a cracking furnace including the cracking zone.

In one embodiment, the light paraffin feedstock comprises ethane, and the cracking conditions in the cracking zone are selected such that 60 wt % or less of ethane in the light paraffin feedstock is converted during a single pass through the cracking zone. It has been found that such low-severity operation of an ethane cracker has particular advantages in an integrated process, leading to a higher overall conversion of feedstock to ethylene and propylene.

The recycle feedstock that is subjected to cracking conditions preferably comprises less than 10 wt % unsaturates, more preferably less that 5 wt %.

In one embodiment the recycle feedstock that is subjected to cracking conditions comprises butane obtained by first selectively hydrogenating a C4 fraction of the combined effluent to obtain a partly hydrogenated C4 effluent, and further hydrogenating the partly hydrogenated C4 effluent to convert butene to butane.

The invention moreover provides an integrated system for the preparation of a olefin product comprising ethylene and/or propylene, which system comprises

a) a steam cracking system having one or more inlets for a paraffin feedstock comprising C2 to C5 paraffins and steam, and an outlet for a cracker effluent comprising C2 to C4 olefins;
b) an oxygenate-to-olefins conversion system, having one or more inlets for receiving an oxygenate feedstock, and comprising a reaction zone for contacting the oxygenate feedstock with an oxygenate conversion catalyst under oxygenate conversion conditions, and an outlet for a conversion effluent comprising C2 to C4 olefins;
c) a work-up section arranged to receive at least part of the cracker effluent and at least part of the conversion effluent to obtain a combined effluent, the work-up section comprising a separation system, an outlet for a lower olefin product stream, and a hydrogenation unit for at least partially hydrogenating at least part of a C4 portion from the combined effluent, an outlet for an at least partially hydrogenated C4 feedstock; and means for recycling at least part of the at least partially hydrogenated C4 feedstock to step the steam cracking system and/or the oxygenate-to-olefins conversion system.

### BRIEF DESCRIPTION OF THE DRAWINGS

The invention will now be discussed by way of example in more detail, with reference to the drawings.

FIGS. 1-5 schematically show various embodiments of an integrated system and process in accordance with the invention.

### DETAILED DESCRIPTION

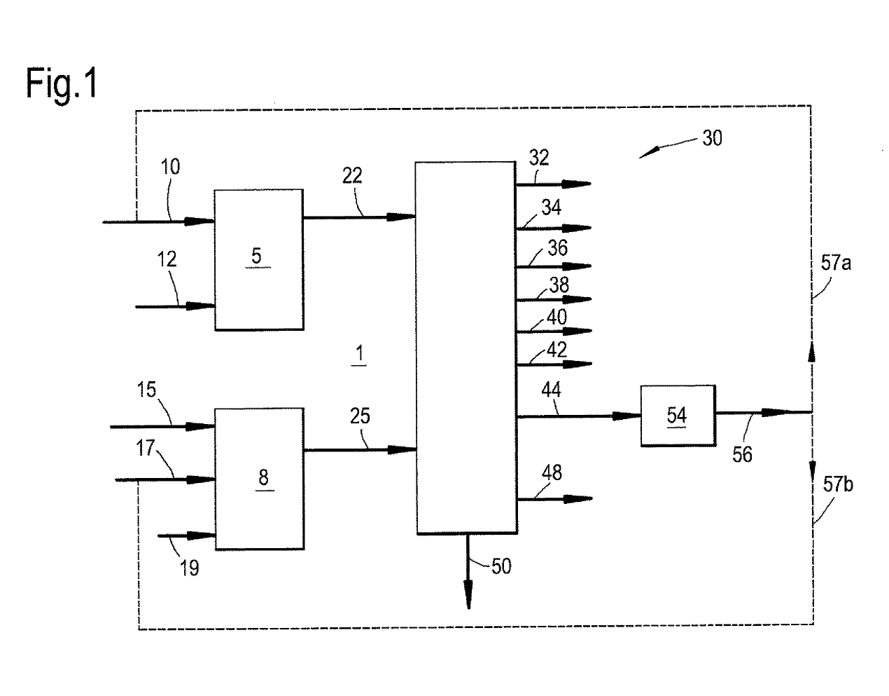

Reference is made to FIG. 1, showing a first embodiment of an integrated system for producing lower olefins for conducting a process in accordance with the invention.

The integrated system 1 comprises a cracking system 5, also referred to as cracker 5, and an oxygenate-to-olefins (OTO) conversion system 8. A light paraffinic feedstock is fed via line 10 to the steam cracker 5. Preferably the cracking system is an ethane cracker, and the light paraffinic feedstock is an ethane-comprising feedstock, and preferably comprises at least 50 wt % ethane, preferably at least 60 wt %, more preferably at least 70 wt %. Ethane-rich feedstock maximises ethylene production. Water or steam is also fed to the cracker 5 via line 12 as diluent. Steam cracking will be discussed in more detail below.

An oxygenate feedstock is fed via line 15 to OTO conversion system 8, for example comprising methanol and/or dimethylether. Optionally, a hydrocarbon stream and/or a diluent are fed to the OTO conversion system via lines 17 or 19, respectively.

In principle every known OTO conversion system and process can be used in conjunction with the present invention, including processes known as Methanol-to-Olefins (MtO) and Methanol to Propylene (MtP). The OTO conversion system and process can for example be a as disclosed in US2005/0038304, incorporated herein by reference, or as disclosed in WO2009/039948, incorporated herein by reference, WO-A 2006/020083 incorporated herein by reference, or as disclosed in any of the publications by Eng, UOP, and Zimmermann identified in the introductory part hereinabove, all incorporated by reference. Other particularly suitable OTO conversion processes and systems with specific advantages are disclosed in WO2007/135052, WO2009/065848, WO2009/065877, and also WO2009/065875, WO2009/065870, WO2009/065855, all incorporated by reference, in which processes a catalyst comprising an aluminosilicate or zeolite having one-dimensional 10-membered ring channels, and an olefinic co-feed and/or recycle feed is employed.

Preferably the OTO conversion system is arranged to receive an olefinic stream, and is able to at least partially convert this stream, in particular a stream comprising C4 olefins, to ethylene and/or propylene. In one option, the recycle feedstock can be contacted with the oxygenate conversion catalyst, in particular as a olefinic co-feed to the oxygenate feed, in the oxygenate-to-olefins reaction zone, see for example WO2009/039948 or WO2007/135052, WO2009/065848, WO2009/065877. The oxygenate conversion catalyst in this case preferably comprises an aluminosilicate, in particular a zeolite. In another option the oxygenate-to-olefins conversion system comprises an olefin cracking zone downstream from the oxygenate-to-olefins reaction zone and arranged to crack C4+ olefins produced in the oxygenate-to-olefins reaction zone, as for example in U.S. Pat. No. 6,809,227 or US2004/0102667, and the recycle feedstock is fed in this option to the olefin cracking zone. Suitable OTO conversion processes and systems will be discussed in more detail below.

In the steam cracker 5 the light paraffinic feedstock is cracked under cracking conditions, to produce a cracker effluent comprising lower olefins in line 22.

In the OTO conversion system 8 the oxygenate feedstock, and optionally an olefinic co-feed (which can be partly or fully a recycle stream), is contacted with an oxygenate conversion catalyst under oxygenate conversion conditions, to obtain a conversion effluent comprising lower olefins in line 25.

Effluents from cracking and oxygenate conversion need to be worked up in order to separate and purify various components as desired, and in particular to separate one or more lower olefins product streams. FIG. 1 shows schematically a common work-up section, which receives and processes at least part of the conversion effluent and at least part of the cracker effluent.

Typically, in known steam cracking as well as OTO processes, the effluent is quenched in a quench unit with quench medium such as water, in order to first cool the process gas close to ambient temperature before it is fed to the compressor, to allow for a smaller compressor frame size and lower power consumption due to reduced gas volume. Other benefits of a quench unit are higher compression efficiency at lower temperature and condensation of water vapour upstream of the compressor jointly with readily condensable hydrocarbon components that are formed in small amounts. Any liquid heavy hydrocarbons are phase separated from liquid water and separately recovered. Water or steam from the quench unit can be partially recycled as diluent to the cracker (line 12) and/or to the OTO conversion system (line 19), optionally after treatment or purification as needed, e.g. to remove catalyst fines. Vapour components after quench are typically sent to a compression section, subjected to a caustic wash treatment, dried, and sent to a separation system including a cold section, so as to obtain separate streams of main components. FIG. 1 shows hydrogen stream 32, light ends stream 34 typically including methane and/or CO, ethane stream 36, ethylene stream 38, propane stream 40, propylene stream 42, a C4 stream 44, a C5+ stream 48, and a water effluent 50. There can also be a separate outlet for heavy (liquid) hydrocarbons (not shown). The C4 stream is a concentrated C4 stream. It will be understood that separation can be conducted differently, such that certain streams are combined, or further separation can be carried out, such as on the C5+ fraction or the C4 stream. For example, a concentrated butadiene stream could be provided such as by butadiene extraction, which allows to process butadiene separately. It is also possible that the C4 stream contains heavier components, such as C5 and/or C6 components. Each stream will have a purity as desired and can contain a certain concentration of other product components as contaminant.

It will be understood, and this is discussed for example in US2005/0038304, that cracker and oxygenate conversion effluents can be combined at various stages of work-up, such as before quenching, after quench and before compression, or after compression. Even if the effluents are combined before quenching, certain process steps such as cooling/heat exchanging can be carried out on one or both effluents separately. Preferably it is not required to include a primary fractionator for heavy components from cracking before a quench tower. This is the case when a sufficiently light paraffin feed is fed to the cracking section.

It can be advantageous to recycle at least part of various streams to either the cracking system 5 and/or the OTO conversion system 8. Ethane from line 36 is preferably recycled as feedstock to the cracker 5 (to line 10), and the ethane comprises ethane cracker feed that was unconverted, as well as ethane contained in the OTO conversion effluent. Optionally also some or all of the propane from line 40 is recycled to the cracker. In this way additional ethylene and propylene is obtained. It can also be desired to recycle part of light ends, an olefinic C4/C4=stream and/or a C5/C6 component to an OTO conversion system.

Typically, both the steam cracker effluent and the OTO conversion system effluent will contain C4 species, in particular including unsaturated C4 species. The cracker effluent will typically contain more butadiene than the reactor effluent. Both effluents typically contain butene, and a quantity of butane.

FIG. 1 shows the C4 stream 44 is being fed to a hydrogenation unit 54. It shall be understood that only part of the C4 species in the combined effluents 22 and 25 need form the stream 44, and that other outlet streams for C4 species can be provided. In a preferred embodiment, substantially all, i.e. 90 wt % or more, of all C4 species are combined in the stream 44. In the unit 54 the stream 44 is hydrogenated with a source of hydrogen, and an at least partially hydrogenated C4 feedstock is obtained in line 56. The hydrogen can at least partially originate from the cracker 5. In accordance with the invention, at least part of the at least partially hydrogenated C4 feedstock is recycled to the cracking system 5 (e.g. combined with line 10 or fed separately into cracker 5) and/or the OTO conversion system 8 (typically via line 17, which can be combined with line 15 and/or steam line 19 before entering the oxygenate conversion zone). Respective options for recycle streams are indicated by dashed lines 57a and 57b. When recycling to the OTO conversion system 8, the recycle stream can be a co-feed to the OTO reaction zone, in an OTO reactor. It can also be a feed to a catalytic olefin cracking zone downstream from the oxygenate-to-olefins reaction zone. Suitable catalysts and conditions are described in U.S. Pat. No. 6,809,227 and US2004/0102667. Catalysts include those comprising zeolitic molecular sieves such as MFI-type, e.g. ZSM-5, or MEL-type, e.g. ZSM-11, as well as Boralite-D and silicalite 2.

In one particular embodiment, the stream 44 comprises a significant quantity of di-olefins, in particular butadiene. A significant quantity of butadiene is for example at least 0.1 wt % of butadiene in the stream, in particular at least 0.5 wt %, more in particular at least 1 wt %, or at least 2 wt %. This is typically the case if the C4 stream 44 comprises substantially all, such as 90 wt % or more, of C4 species in the combined effluents 22,25. Cracker effluent is typically much richer in butadiene than OTO conversion effluent. In particular embodiments stream 44 can also be a butadiene-enriched stream such as resulting from a butadiene extraction.

The stream comprising a significant quantity of butadiene is subjected to selective hydrogenation conditions in hydrogenation unit 54 in the presence of hydrogen and a hydrogenation catalyst, to convert butadiene to butene, but preferably minimizing the hydrogenation of butene (contained in the C4 stream and/or hydrogenation products from butadiene) to butane.

Suitable processes for such selective hydrogenation (also referred to as partial, mild or semi-hydrogenation) are known in the art, and reference is made by way of example to Derrien, M. L. “Selective hydrogenation applied to the refining of petrochemical raw materials produced by steam cracking” (1986) Stud. Surf. Sci. Catal., 27, pp. 613-666, to WO 95/15934, or to U.S. Pat. No. 4,695,560. Typically at least 90 wt % of butadiene is converted to butene, and less than 10 wt %, preferably less than 5 wt %, of the butene, based on butene in the feed to the selective hydrogenation, is converted to butane.

The effluent from selective hydrogenation is a C4 feedstock comprising butene, and butene is a desirable co-feed in oxygenate-to-olefins reactions, in particular in the MTP process or in a process in which a catalyst comprising an aluminosilicate or zeolite having one-dimensional 10-membered ring channels, and an olefinic co-feed and/or recycle feed is employed, and is recycled via line 57b.

In another embodiment, stream 44 comprising unsaturated C4 species is subjected to more severe hydrogenation conditions in unit 54, in the presence of hydrogen and a hydrogenation catalyst, so that butenes as well as any butadiene are substantially fully hydrogenated, and an effluent rich in butane is obtained in line 56. Such a butane-rich stream can be recycled as feedstock to the steam cracker 5 via line 57a, so as to obtain additional ethylene and/or propylene.

Substantially full hydrogenation is whereby the C4 reactor effluent contains an olefin concentration of 1 wt % or less, preferably 0.1 wt % or less, based on total hydrocarbons in the effluent, and is achieved by adjusting the hydrogenation conditions such as partial pressure ratio of hydrogen to olefins in the hydrogenation reactor. When feeding a C4 portion or feedstock to a cracker, it is preferred that the C4 portion or feedstock is substantially fully hydrogenated.

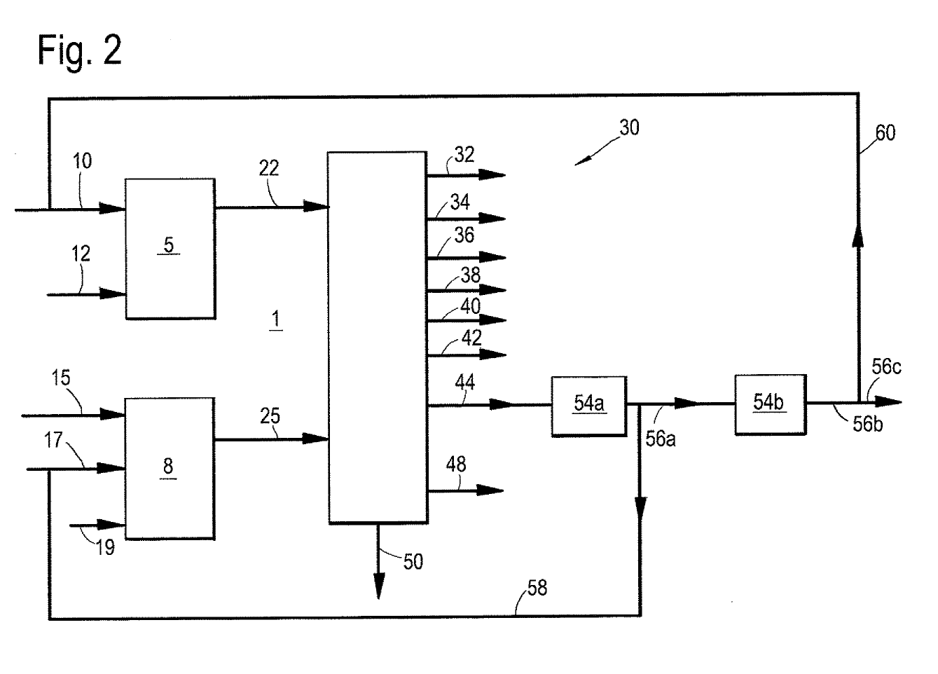

In yet another embodiment, shown in FIG. 2, hydrogenation is carried out in two steps. A first selective hydrogenation step in a first zone 54a is used to hydrogenate butadiene to butene, and part of the butene-enriched effluent 56a is recycled as co-feed via line 58 to the OTO conversion unit 8. The remainder is subjected to severe hydrogenation in second zone 54b, and a butane-rich stream is obtained in line 56b and at least partially recycled via line 60 to the steam cracker 5. A bleed stream or product stream 56c can also be withdrawn, and can for example be sent to an LPG pool. It shall be clear that if desired, bleed or product streams can also be withdrawn if desired from lines 44 or 56a (not shown).

In one preferred embodiment the cracking step is conducted at low severity. Severity is suitably defined by the conversion of ethane in the low paraffin feedstock in a single pass through the steam cracker, i.e. the ratio (by weight) of ethane cracked to other products and therefore not present in the effluent, compared with the ethane in the low paraffin feedstock. A severity of less than 65 wt % ethane conversion is regarded low, in particular a severity of 60 wt % or less, and a very low severity is 50 wt % or less. It is known in the art how to adjust severity (conversion), which is mainly dependent on steam dilution, coil outlet pressure and residence time in the cracking zone, as well as temperature.

Operating the steam cracker at low severity has a number of advantages. At low severity, the selectivity to ethylene is higher, and less by-products are formed. This is desirable in order to convert the valuable ethane feedstock to maximum ethylene. Another advantage is that the total selectivity to C2-C4 lower olefins increases with decreasing severity, and that formation of by-products like methane and C5+ decreases. When butylenes can be recycled, in particular to the OTO step, the net conversion to ethylene and propylene is maximised. In a stand-alone ethane cracker, a disadvantage of operating at lower severity is that the effluent contains a relatively large amount of ethane that needs to be separated and recycled to the cracker, increasing the fuel and capital requirement per ton of ethylene. In an integrated system of steam cracker and OTO conversion reactor, however, this can at least partly be compensated by the fact that OTO conversion effluents have typically a higher concentration of ethylene compared to the concentration of ethane in the cracker effluent. The combined effluent therefore has a higher ethylene/ethane molar ratio than the cracker effluent.

That can be a particular advantage when a steam cracker plant is revamped by adding an OTO conversion system. When, for example, ethane feedstock supply to the cracker diminishes, such as due to depletion of a natural or associated gas source, lower severity operation first of all increases the ethylene yield and at least partially compensates for a loss of ethane feedstock. In addition, synergy benefits are obtained from the integration with an OTO conversion system. For a stand-alone cracking system in a situation of lower ethane feed supply, the product work-up section would not be used to full capacity, since less total feedstock would be processed. Moreover, the ratio of valuable products to unconverted feedstock being processed in the work-up section would change unfavourably when running at lower severity, increasing the work-up cost per ton of product. With the OTO conversion system being integrated, a combined work-up section can operate at higher ratios of products to unconverted feedstock. In addition, capacity that has become available in the work-up section of the cracker can be used to also work up products from the OTO conversion system. Lowering the severity of the steam cracker operation creates some room for OTO conversion effluent to be combined with the cracker effluent for work-up in the existing work-up section of the ethane cracker, wherein it is known that the compression and cold section parts are the most capital intensive and therefore typically most limiting elements.

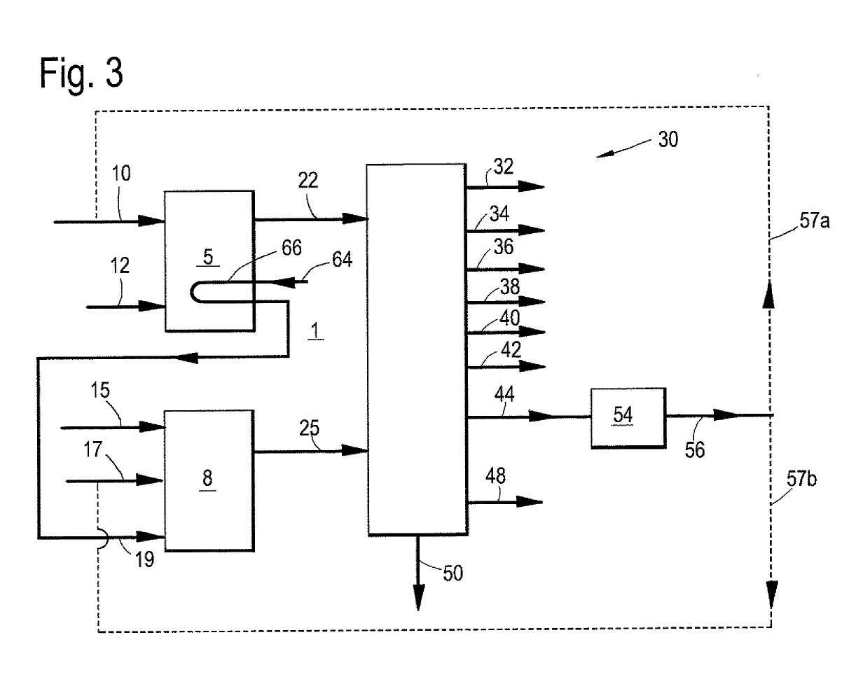

Another disadvantage of operating a stand-alone ethane cracker at lower severity is that the energy consumption for firing of the furnace per unit of ethylene product increases, due to the larger recycle of unconverted ethane. In an integrated system, however, that creates room for beneficial energy integration. In one embodiment, superheated steam needed for addition to the OTO conversion system (line 19) can be generated in the convection section of the cracking furnace. In this case no separate fired heater for OTO steam generation or superheating is required. Ethane furnaces are typically provided with tube banks in the convection section for generating superheated high-pressure steam, such as at 110 bar, by superheating high pressure steam generated outside the furnace in the transfer line exchanger. OTO conversion systems typically require low pressure steam (less than 5 bar), therefore a low-pressure tube bank would suitably be installed in the convection section to produce steam at a temperature and pressure as required in the OTO conversion system. This option is shown in FIG. 3, where a low temperature/low pressure steam is fed via line 64 to bank 66, so that steam as needed at line 19 is produced.

Yet a further advantage of operating the steam cracker at low severity is obtained in case a C4 feedstock, in particular butane, is co-fed with ethane to the steam cracker. The butane can be a recycle stream, but can in principle as well come from an external source. The product distribution from butane cracking changes beneficially at lower severity. Generally, it is well known that butane cracks easier than ethane under the same cracking conditions, i.e. butane conversion is generally higher than ethane conversion. The cracking products from butane are more diverse than cracking products of ethane. Lower severity improves lower olefins yield from butane cracking, in particular the combined ethylene+propylene+butylene yield is increased and by-product formation such as methane and C5+ is decreased. This has the additional benefit of reducing heavy hydrocarbon load in the sump of the quench unit, which typically limits C4 intake in the absence of a primary fractionator. This is a particular advantage when butylenes are recycled to the OTO conversion system for conversion to further ethylene and propylene. These advantages are most pronounced for iso-butane. Iso-butane has a tendency to crack into propylene and (undesired) methane. At lower severity, this cracking reaction occurs relatively less, and relatively more iso-butylene is formed, which can be processed with advantage in an OTO reaction step.

These advantages can already be realised if the severity of butane cracking is such that butane conversion is 98 wt % or less, in particular 97 wt % or less, more in particular 95 wt % or less. Such butane cracking severities can be obtained concurrently with applying low ethane severity as discussed above. Even more pronounced advantages can be obtained at low butane conversions of 90 wt % or less, 75 wt % or less, 60 wt % or less, in particular 50 wt % or less. Severity of butane cracking is suitably defined by the conversion of butane feedstock in a single pass through the cracking zone.

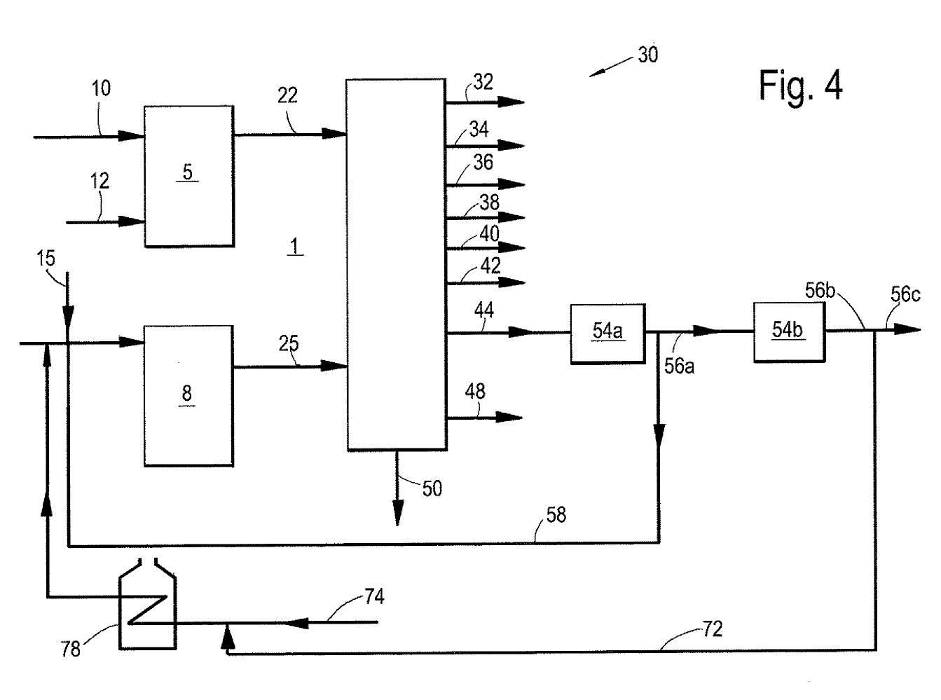

Such low conversion of butane is difficult to realize in a co-feeding situation with ethane to a cracking furnace, since ethane conversion will be substantially lower, so that the advantages from low severity operation do not outweigh the additional effort for ethane recycling. Therefore, in a particular embodiment, a dedicated cracking unit for operating at low butane cracking severity is arranged, which can be operated at a desired butane conversion, e.g. at 90 wt % or lower as discussed above. The effluent from such a low severity butane cracker can in a particular embodiment be fed directly into the OTO conversion unit. FIG. 4 shows a particular way of implementing this. The embodiment in FIG. 4 also includes two hydrogenation zones 54a and 54b like the embodiment of FIG. 2. At least part of the effluent 56b from the second hydrogenation zone 54b is mixed via line 72 with steam in line 74 before that steam is superheated in superheating furnace 78 to the desired temperature for feeding towards the OTO conversion system. The superheating furnace thus can act as steam cracker for the butane-rich effluent. It can heat the mixture comprising steam and butane to e.g. 650° C. or higher, in particular 700° C. or higher, such as 740° C. for example. Dependent on the residence time in the furnace, the conversion of butanes may for example be in the range of from 10-70 wt %, in particular 20-60 wt %. If such operation is desired, it is preferred to use a separate superheating furnace as shown in FIG. 4, instead of a superheater integrated in the convection section of the steam cracker furnace as discussed with reference to FIG. 3. Butane cracking is likely to produce some coke that typically will need to be removed periodically, which is more difficult in a convection bank than in a stand-alone furnace.

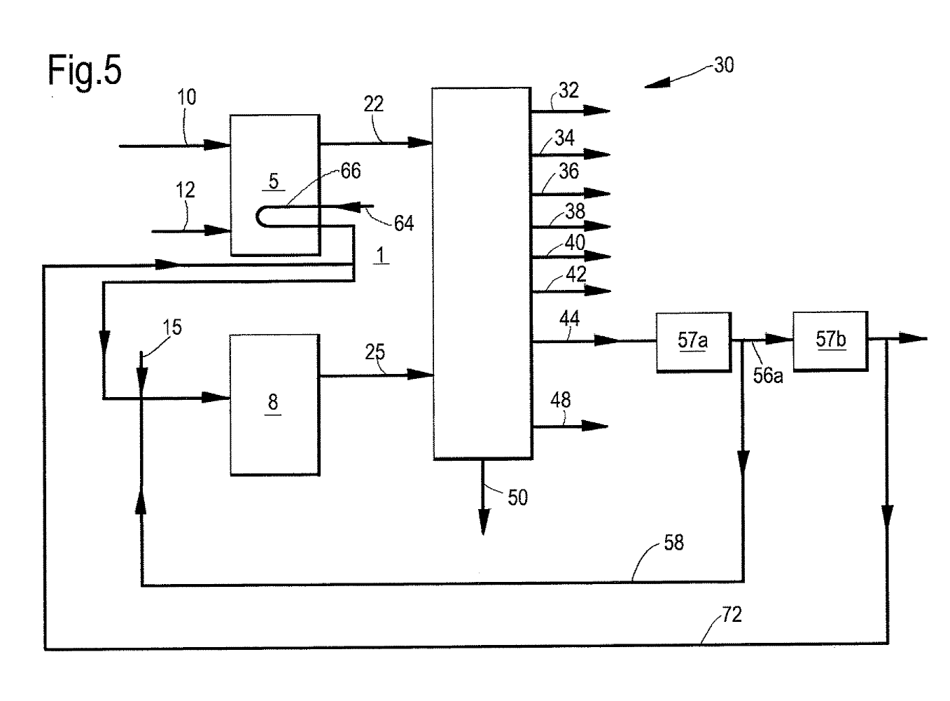

Alternatively to mixing the butane-rich feedstock from line 56b to steam before superheating, it can also be mixed with the superheated steam, i.e. after superheating to e.g. 650° C. or higher, in particular 700° C. or higher, such as 740° C. or higher, which temperatures will also provide mild cracking conditions for the butane. This may limit residence time, coke lay down due to hot spots in the radiant section of the furnace, and may lower the conversion of butanes to 30 wt % or less, such as to 15-30 wt %. It also cools the dilution steam somewhat due to the endothermic nature of C4 cracking. For this operation, a separate superheating furnace as shown in FIG. 4 or a superheating in the steam cracking furnace convection section can be used. The latter option is depicted in FIG. 5. Typically the butane-rich stream will be much smaller than the steam, so that butane will be very diluted in the steam, such as butane:steam<1:10 w/w. A recycling of butane-rich feed to the OTO conversion step via a mild hydrocracking step (such as in combination with superheating steam as discussed) is beneficial as long as more C4 saturates are cracked than are formed in the OTO conversion system per pass, since in this case build-up of C4 saturates is prevented.

Propane can also be recycled for additional yield of lower olefins to the cracking zone, in particular when butane is recycled as well. In the feed to the low paraffin cracking system, the propane can for example be present in an amount of 1-50 wt %. Butane is preferably present in an amount of 15 wt % or less, preferably 10 wt % or less, based on total hydrocarbons in the feed, since otherwise it may be required to provide a primary fractionator for separating heavy cracking products.

It is possible to use a cracking system in an integrated process with a plurality of cracking furnaces, wherein at least two furnaces are operated at different severities. The cracking system can include a first furnace for a relatively lighter feedstock portion and a second furnace for a relatively heavier feedstock portion, the first and second furnaces can be at selected different severities adapted to the type of feed. It is for example possible to assign different feeds to various furnaces of a stream cracking system, such as one, two or more dedicated furnaces for ethane, propane, butane, and/or particular mixtures. This allows selecting of the severity individually for different parts of the low-paraffin feedstock. The dedicated butane furnace for example could be operated at a butane conversion of 90 wt % or lower, such as 50 wt %, all of which would be lower severities than e.g. the severity of an ethane furnace running at 60 wt % ethane conversion. Also a propane furnace could operate at lower severity than in a co-feeding situation with ethane, such as at 90 wt % propane conversion or less (defined analogous to ethane conversion), at 85 wt % or less, or at 80 wt % or less, in order to increase selectivity to ethylene.

An additional benefit of the present invention is that the production of hydrogen increases. Hydrogen produced in an integrated cracker and an OTO conversion system can be used for selective and/or full hydrogenation, but also in the synthesis of oxygenates such as methanol or dimethylether.

### EXAMPLES

In Examples 1-6, several options of implementing the present invention are compared with comparative examples, by means of model calculations. As basis for Examples 1-6 a model integrated OTO/ethane cracker process was taken. In Table 1, an overview is provided of the feed input and the calculated products.

Calculations were done using a Spyro based model for modelling of cracker operation combined with a proprietary model for modelling the OTO conversion. The key input to the models was as follows:

Cracking:

Steam to ethane ratio is 0.35 wt %. USC coil is used for the Spyro calculations. Calculated at a coil outlet pressure of 1.77 bar absolute, at 65% ethane conversion and a residence time of 0.24 seconds.

OTO Conversion:

MeOH 5012 t/d is fed to the OtO reactor together with 1384 t/d of recycled and superheated steam and recycle of C4 components, in the comparative base case without cracker integration (example 1) 1775 t/d of recycled C4 stream. The model was calibrated on small-scale experiments conducted to determine product distributions for single-pass OTO conversions. Therein, all components that were fed to the OTO reactor have been evaporated and heated such that the temperature in the reactor is controlled between 550-600° C. The pressure in the reactor is 2 bar absolute. The OTO catalyst is fluidized in the reaction medium under the condition that the weight hourly space velocity (WHSV) is 4-10 h⁻¹, whereby WHSV is defined as the total weight of the feed flow over the catalyst weight per hour. The following catalyst was used: Composition and preparation: 32 wt % ZSM-23 SAR 46, 8 wt % ZSM-5 SAR 280, 36 wt % kaolin, 24 wt % silica sol, and, after calcination of the ammonium form of the spray dried particle, 1.5 wt % P was introduced by H₃PO₄ impregnation. The catalyst was again calcined at 550 C. The steam and C4 recycle streams are excluded from the product composition tables.

Table 1 summarizes the net product distribution from a feed of 5012 t/d methanol to an OTO conversion system and/or 2755 t/d ethane to a steam cracker. The yields are calculated on a weight basis as the yield of ethylene and propylene based on CH₂ in the feed.

### Example 1 (Comparative)

In this example the net product distribution of an OTO conversion unit with a net feed intake of 5012 t/d methanol and with internal recycle of C4 product as co-feed to the oxygenate conversion zone is shown. The C4 recycle stream is 1775 t/d. The C4 net product shown in Table 1 is the C4 purge stream that is withdrawn as an outlet for butane produced which would otherwise build up in the process. The ratio of butane/butene is 3:1.

### Example 2 (Comparative)

In this example the net product distribution of cracking 2755 t/d ethane is shown. Unconverted ethane is recycled. The negative water make reflects the make up for losses in the circulation of steam. Due to its low amount, propene from a cracker can often not be economically be recovered and is wasted, in which case the yield should rather be calculated on ethylene only basis to 85.1%.

### Example 3 (Comparative)

In this example an OTO conversion system and a steam cracking system are operated together, each as in Examples 1 and 2, and only integrated regarding the recycle of ethane produced in the OTO system to the cracker. This can be achieved by separate product work-up sections, or if a common work-up section is used the butadiene is extracted before recycling C4 to the OTO conversion system.

### Example 4

In this example according to the invention, butadiene is selectively hydrogenated (either as part of a mixed C4 stream or after extraction), providing additional butene that is being recycled to the OTO conversion system. This increases the yield of valuable ethylene and propylene, whereas there is normally no economic outlet to higher value products for such a relatively low quantity of butadiene. The purge stream of butene and butane is kept the same as the sum of butane+butene streams of examples 1 and 2.

### Example 5

Building on example 4, the remaining stream of butane+butene is not purged but fully hydrogenated and cracked at low severity with a butane conversion of 50 wt % before recycling to the OTO conversion system, such as discussed with reference to FIG. 4 or 5, e.g. in a steam superheater furnace. The yield of ethylene and propylene increases further, whereas there is normally no economic outlet to higher value products for such a relatively low quantity of mixed C4's.

### Example 6

Building on example 4, the remaining stream of butane+butene is not purged but fully hydrogenated and recycled to the cracking system, such as discussed with reference to FIG. 2. The yield of ethylene and propylene increases with respect to example 4, and is comparable to that of example 5, with a slightly different distribution of ethylene and propylene.

TABLE 1

| | Example: | | |
|---|---|---|---|
| | 1 | 2 | 3 |
| | 10³ kg/day | 10³ kg/day | 10³ kg/day |
| Feed: | | | |
| Methanol | 5012 | | 5012 |
| Ethane | | 2755 | 2755 |
| Products: | | | |
| Ethylene | 512 | 2187 | 2713 |
| Propylene | 1275 | 50 | 1325 |
| Ethane | 17 | 0 | 0 |
| Propane | 48 | 19 | 67 |
| C4: | | | |
| Butane + Butene | 35.9 | 22 | 58 |
| Butadiene | 0.3 | 52 | 52 |
| C5+ | 282 | 61 | 343 |
| Fuel gas | 98 | 197 | 280 |
| H₂O | 2710 | −83 | 2627 |
| Hydrogen | 17 | 168 | 186** |
| Yield (wt % CH2) | 81.5% | 87.1 | 84.8% |
| | Example: | | |
| | 4 | 5 | 6 |
| | 10³ kg/day | 10³ kg/day | 10³ kg/day |
| Feed: | | | |
| Methanol | 5012 | 5012 | 5012 |
| Ethane | 2755 | 2755 | 2755 |
| Products: | | | |
| Ethylene | 2725 | 2738 | 2743 |
| Propylene | 1356 | 1383 | 1371 |
| Ethane | 0 | 0 | 0 |
| Propane | 68 | 68 | 68 |
| C4: | | | |
| Butane + Butene | 58 | 0 | 0 |
| Butadiene | 0 | 0 | 0 |
| C5+ | 350 | 355 | 361 |
| Fuel gas | 282 | 295 | 296 |
| H₂O | 2627 | 2627 | 2627 |
| Hydrogen | 186 | 187 | 187 |
| Yield (wt % CH2) | 85.7 | 86.5 | 86.4 |

### Example 7

In this example the effect of operating an ethane cracker at lower severity is shown. Selectivity of cracking to various products in a single pass through the cracking zone is shown in Table 2. All selectivity data represent the weight percentage of the respective component in the cracking products, based on total products not counting unconverted ethane. Clearly, the selectivity to ethylene, propylene, and butylene increases with decreasing severity, wherein butylenes can be further converted to ethylene and propylene in accordance with the present invention. An ethane conversion of 65 wt % is known from the article “Integration of the UOP/HYDRO MTO Process into Ethylene plants” by C. N. Eng et al., referenced above.

TABLE 2

Ethane
Conversion (wt %)

| | 40.0 | 50.0 | 55.0 | 70.0 |
|---|---|---|---|---|
| Ethylene | 85.9 | 83.7 | 82.4 | 77.8 |
| Selectivity | | | | |
| Hydrogen | 6.4 | 6.4 | 6.4 | 6.3 |
| Selectivity | | | | |
| Methane | 3.0 | 3.9 | 4.4 | 7.6 |
| Selectivity | | | | |
| Propylene | 1.0 | 1.3 | 1.4 | 1.6 |
| Selectivity | | | | |
| Butylene | 1.5 | 1.9 | 2.2 | 2.9 |
| Selectivity | | | | |
| C2 = +C3 = +C4 = | 88.4 | 86.9 | 86.0 | 82.3 |
| Selectivity | | | | |
| C5+ make per | 0.82 | 1.59 | 2.07 | 4.18 |
| ton olefin | | | | |

### Example 8

In this example the effect of operating the ethane cracker at low severity on the conversion of co-fed butane is shown. Selectivity of cracking of co-fed butane (50% n-butane and 50% iso-butane) in a single pass through the cracking zone to various products is shown in Table 3. All selectivity data represent the weight percentage of the respective component in the cracking products, based on total products not counting unconverted butane. It can be seen that at lower severity more total C2-C4 lower olefins are produced from butane, and less by-products methane, C5+ are formed.

TABLE 3

| | | | |
|---|---|---|---|
| Ethane Conversion | 50.0 | 60.0 | 65.0 |
| (wt %) | | | |
| Butane Conversion | 93.9 | 97.8 | 98.9 |
| (wt %) | | | |
| Ethylene | 36.4% | 38.0% | 38.4% |
| Selectivity | | | |
| Methane | 6.5% | 7.3% | 7.7% |
| Selectivity | | | |
| Propylene | 14.2% | 10.2% | 8.3% |
| Selectivity | | | |
| Butylene | 7.4% | 5.7% | 5.0% |
| Selectivity | | | |
| C2 = +C3 = +C4 = | 58.0% | 54.0% | 51.8% |
| Selectivity | | | |
| C5+ make per ton | 5.42% | 6.77% | 7.38% |
| olefin | | | |

### Example 9

In this example the product distribution of cracking n-butane and iso-butane under low severity conditions is shown. Selectivity of cracking of n-butane and iso-butane, respectively at 50 wt % and 90 wt % conversion in a single pass through the cracking zone to various products is shown in Table 4. All selectivity data represent the weight percentage of the respective component in the cracking products, based on total products not counting unconverted butane.

The data show that the selectivity to ethylene, propylene, and butylene increases with decreasing severity, wherein butylenes can be further converted to ethylene and propylene in accordance with the present invention. Moreover, iso-butane selectivity to butylene increases significantly, which is valuable recycle feedstock to the OTO conversion system. Methane on the other hand is significantly reduced. Iso-butane cracking, which at higher conversions produces less of the valuable lower olefins, produces at 50% selectivity more ethylene propylene and butylenes (C2=, C3=, C4=) than n-butane, which is of particular advantage on the integrated process.

TABLE 4

| | |
|---|---|
| Iso-butane | n-butane |

| | | | | |
|---|---|---|---|---|
| Butane | 50.0% | 95.0% | 50.0% | 95.0% |
| Conversion | | | | |
| Ethylene | 4.8% | 14.0% | 31.1% | 42.6% |
| Selectivity | | | | |
| Hydrogen | 1.4% | 1.3% | 0.8% | 1.2% |
| Selectivity | | | | |
| Methane | 17.6% | 25.6% | 18.8% | 21.7% |
| Selectivity | | | | |
| Propylene | 32.1% | 21.6% | 37.0% | 16.0% |
| Selectivity | | | | |
| Butylene | 37.4% | 17.7% | 4.8% | 5.8% |
| Selectivity | | | | |
| C2 = +C3 = +C4 = | 74.3% | 53.2% | 72.9% | 64.5% |
| Selectivity | | | | |

In the present invention a light paraffin feedstock is cracked in a cracking zone under cracking conditions to produce at least olefins and hydrogen.

Preferably the cracking system is an ethane cracker, and the light paraffinic feedstock is a feedstock comprising C2-C5 paraffins, in particular C2-C4 paraffins, i.e. comprises one or more of ethane, propane, a mixed C4 stream or a mixture comprising two or more thereof. The light paraffinic feedstock is preferably an ethane-comprising feedstock, and preferably comprises at least 35 wt % ethane, more preferably at least 50 wt %, more preferably at least 70 wt %. Ethane-rich feedstock maximises ethylene production. Although not normally preferred, other hydrocarbons such as olefins can also be contained in the light paraffin feedstock, preferably in quantities of less than 10 wt % based on total hydrocarbons. The light paraffin feed may comprise a recycle stream from the process.

Preferably, the ethane-comprising feedstock is obtained from natural gas or associated gas.

The cracking process is performed at elevated temperatures, preferably in the range of from 650 to 1000° C., more preferably of from 750 to 950° C.

Steam is usually added to the cracking reactor, acting as a diluent reducing the hydrocarbon partial pressure and thereby enhancing olefin yield. Steam also reduces the formation and deposition of carbonaceous material or coke in the cracking reactors. The process is also referred to as steam cracking or pyrolysis.

Such cracking processes are well known to the skilled person and need no further explanation. Reference is for instance made to Kniel et al., Ethylene, Keystone to the petrochemical industry, Marcel Dekker, Inc, New York, 1980, in particular chapter 6 and 7, as well as to US2005/0038304, WO2009/039948, or the publications by Eng, UOP, and Zimmermann identified in the introductory part hereinabove.

In addition to ethylene and some propylene, other by-products are formed as well. By-products can include butylene, butadiene, ethyne, propyne and benzene. Coke may also be formed and may require regular cleaning of the steam cracker furnace such as through decoking with air.

In step b) of the process the present invention an oxygenate feedstock is converted in an oxygenate-to-olefins conversion system, in which an oxygenate feedstock is contacted in a reaction zone with an oxygenate conversion catalyst under oxygenate conversion conditions, to obtain a conversion effluent comprising lower olefins. Oxygenates in the oxygenate feedstock are converted to olefins, in particular, ethylene and/or propylene. In addition, any olefins in the oxygenate feed may be cracked to smaller olefins, in particular ethylene and/or propylene. In the OTO reaction zone, at least part of the feed is converted into olefins to obtain a product containing one or more olefins, preferably including light olefins, in particular ethylene and/or propylene.

Examples of oxygenates that can be used in the oxygenate feedstock of step b) of the process include alcohols, such as methanol, ethanol, isopropanol, ethylene glycol, propylene glycol; ketones, such as acetone and methylethylketone; aldehydes, such as formaldehyde, acetaldehyde and propionaldehyde; ethers, such as dimethylether, diethylether, methylethylether, tetrahydrofuran and dioxane; epoxides such as ethylene oxide and propylene oxide; and acids, such as acetic acid, propionic acid, formic acid and butyric acid. Further examples are dialkyl carbonates such as dimethyl carbonate or alkyl esters of carboxylic acids such as methyl formate. Of these examples, alcohols and ethers are preferred.

Examples of preferred oxygenates include alcohols, such as methanol, ethanol, isopropanol, ethylene glycol, propylene glycol; and dialkyl ethers, such as dimethylether, diethylether, methylethylether. Cyclic ethers such as tetrahydrofuran and dioxane, are also suitable.

The oxygenate used in the process according to the invention is preferably an oxygenate which comprises at least one oxygen-bonded alkyl group. The alkyl group preferably is a C1-C4 alkyl group, i.e. comprises 1 to 4 carbon atoms; more preferably the alkyl group comprises 1 or 2 carbon atoms and most preferably one carbon atom. The oxygenate can comprise one or more of such oxygen-bonded C1-C4 alkyl groups. Preferably, the oxygenate comprises one or two oxygen-bonded C1-C4 alkyl groups.

More preferably an oxygenate is used having at least one C1 or C2 alkyl group, still more preferably at least one C1 alkyl group.

Preferably the oxygenate is chosen from the group of alkanols and dialkyl ethers consisting of dimethylether, diethylether, methylethylether, methanol, ethanol and isopropanol, and mixtures thereof.

Most preferably the oxygenate is methanol or dimethylether, or a mixture thereof.

Preferably the oxygenate feedstock comprises at least 50 wt % of oxygenate, in particular methanol and/or dimethylether, based on total hydrocarbons, more preferably at least 80 wt %, most preferably at least 90 wt %.

The oxygenate feedstock can be obtained from a prereactor, which converts methanol at least partially into dimethylether. In this way, water may be removed by distillation and so less water is present in the process of converting oxygenate to olefins, which has advantages for the process design and lowers the severity of hydrothermal conditions the catalyst is exposed to.

The oxygenate feedstock can comprise an amount of diluents, such as water or steam.

In one embodiment, the oxygenate is obtained as a reaction product of synthesis gas. Synthesis gas can for example be generated from fossil fuels, such as from natural gas or oil, or from the gasification of coal. Suitable processes for this purpose are for example discussed in Industrial Organic Chemistry, Klaus Weissermehl and Hans-Jürgen Arpe, 3rd edition, Wiley, 1997, pages 13-28. This book also describes the manufacture of methanol from synthesis gas on pages 28-30.

In another embodiment the oxygenate is obtained from biomaterials, such as through fermentation. For example by a process as described in DE-A-10043644.

Preferably, at least part of the oxygenate feed is obtained by converting methane into synthesis gas and providing the synthesis gas to a oxygenate synthesis zone to synthesise oxygenates. The methane is preferably obtained from natural gas or associated gas, more preferably the same natural gas or associated gas, from which the light paraffin feedstock for the cracker is obtained.

The oxygenate feedstock may be provided directly from one or more oxygenate synthesis zones, however, it may also be provided from an oxygenate storage facility.

A variety of OTO processes are known for converting oxygenates such as for instance methanol or dimethylether to an olefin-containing product, as already referred to above. One such process is described in WO-A 2006/020083, incorporated herein by reference, in particular in paragraphs [0116]-[0135]. Processes integrating the production of oxygenates from synthesis gas and their conversion to light olefins are described in US2007/0203380A1 and US2007/0155999A1.

Catalysts as described in WO A 2006/020083 are suitable for converting the oxygenate feedstock in step (b) of the present invention. Such catalysts preferably include molecular sieve catalyst compositions. Suitable molecular sieves are silicoaluminophosphates (SAPO), such as SAPO-17, -18, -34, -35, -44, but also SAPO-5, -8, -11, -20, -31, -36, -37, -40, -41, -42, -47 and -56.

Alternatively, the conversion of the oxygenate feedstock may be accomplished by the use of an aluminosilicate catalyst, in particular a zeolite. Suitable catalysts include those containing a zeolite of the ZSM group, in particular of the MFI type, such as ZSM-5, the MTT type, such as ZSM-23, the TON type, such as ZSM-22, the MEL type, such as ZSM-11, the FER type. Other suitable zeolites are for example zeolites of the STF-type, such as SSZ-35, the SFF type, such as SSZ-44 and the EU-2 type, such as ZSM-48. Aluminosilicate catalysts are preferred when an olefinic co-feed is fed to the oxygenate conversion zone together with oxygenate, for increased production of ethylene and propylene.

The reaction conditions of the oxygenate conversion include those that are mentioned in WO-A 2006/020083. Hence, a reaction temperature of 200 to 1000° C., preferably from 250 to 750° C., and a pressure from 0.1 kPa (1 mbar) to 5 MPa (50 bar), preferably from 100 kPa (1 bar) to 1.5 MPa (15 bar), are suitable reaction conditions.

A specially preferred OTO process for use in step (b) of the present invention will now be described. This process provides particularly high conversion of oxygenate feed and a recycle co-feed to ethylene and propylene. Reference is made in this regard also to WO2007/135052, WO2009/065848, WO2009/065875, WO2009/065870, WO2009/065855, WO2009/065877, in which processes a catalyst comprising an aluminosilicate or zeolite having one-dimensional 10-membered ring channels, and an olefinic co-feed and/or recycle feed is employed.

In this process, the oxygenate-conversion catalyst comprises one or more zeolites having one-dimensional 10-membered ring channels, which are not intersected by other channels, preferably at least 50% wt of such zeolites based on total zeolites in the catalyst. Preferred examples are zeolites of the MTT and/or TON type. In a particularly preferred embodiment the catalyst comprises in addition to one or more one-dimensional zeolites having 10-membered ring channels, such as of the MTT and/or TON type, a more-dimensional zeolite, in particular of the MFI type, more in particular ZSM-5, or of the MEL type, such as zeolite ZSM-11. Such further zeolite (molecular sieve) can have a beneficial effect on the stability of the catalyst in the course of the OTO process and under hydrothermal conditions. The second molecular sieve having more-dimensional channels has intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably the channels in at least one of the directions are 10-membered ring channels. A preferred MFI-type zeolite has a Silica-to-Alumina ratio SAR of at least 60, preferably at least 80, more preferably at least 100, even more preferably at least 150. The oxygenate conversion catalyst can comprise at least 1 wt %, based on total molecular sieve in the oxygenate conversion catalyst, of the second molecular sieve having more-dimensional channels, preferably at least 5 wt %, more preferably at least 8 wt %, and furthermore can comprise less than 35 wt % of the further molecular sieve, in certain embodiments less than 20 wt %, or less than 18 wt %, such as less than 15 wt %.

Especially when the oxygenate conversion is carried out over a catalyst containing MTT or TON type aluminosilicates, it may be advantageous to add an olefin-containing co-feed together with the oxygenate feed (such as dimethylether-rich or methanol-rich) feed to the reaction zone when the latter feed is introduced into this zone. It has been found that the catalytic conversion of the oxygenates, in particular methanol and DME, to ethylene and propylene is enhanced when an olefin is present in the contact between methanol and/or dimethylether and the catalyst. Therefore, suitably, an olefinic co-feed is added to the reaction zone together with the oxygenate feedstock.

In special embodiments, at least 70 wt % of the olefinic co-feed, during normal operation, is formed by a recycle stream of a C3+ or C4+ olefinic fraction from the OTO conversion effluent or the combined OTO conversion and cracker effluents, preferably at least 90 wt %, more preferably at least 99 wt %, and most preferably the olefinic co-feed is during normal operation formed by such recycle stream. Preferably this recycle stream is obtained from the combined effluent and at least partially hydrogenated in accordance with the present invention. In one embodiment the olefinic co-feed can comprise at least 50 wt % of C4 olefins, and at least a total of 70 wt % of C4 hydrocarbon species. It can also comprise propylene. The OTO conversion effluent can comprise 10 wt % or less, preferably 5 wt % or less, more preferably 1 wt % or less, of C6-C8 aromatics, based on total hydrocarbons in the effluent. At least one of the olefinic co-feed, and the recycle stream, can in particular comprise less than 20 wt % of C5+ olefins, preferably less than 10 wt % of C5+ olefins, based on total hydrocarbons in the olefinic co-feed.

In order to maximize production of ethylene and propylene, it is desirable to maximize the recycle of C4 olefins. In a stand-alone process, i.e. without integration with a cracker, there is a limit to the maximum recycle of a C4 fraction from the OTO effluent. A certain part thereof, such as between 1 and 5 wt %, needs to be withdrawn as purge, since otherwise saturated C4's (butane) would build up which are substantially not converted under the OTO reaction conditions. In the integration with a cracker however, more C4 olefins are available and can be provided by hydrogenating butadiene, moreover any stream previously purged can be recycled to the cracker, preferably after full hydrogenation.

In the preferred process, optimum light olefins yield are obtained when the OTO conversion is conducted at a temperature of more than 450° C., preferably at a temperature of 460° C. or higher, more preferably at a temperature of 480° C. or higher, in particular at 500° C. or higher, more in particular 550° C. or higher, or 570° C. or higher. The temperature will typically be less than 700° C., or less than 650° C. The pressure will typically be between 0.5 and 15 bar, in particular between 1 and 5 bar.

In a special embodiment, the oxygenate conversion catalyst comprises more than 50 wt %, preferably at least 65 wt %, based on total molecular sieve in the oxygenate conversion catalyst, of the one-dimensional molecular sieve having 10-membered ring channels.

In one embodiment, molecular sieves in the hydrogen form are used in the oxygenate conversion catalyst, e.g., HZSM-22, HZSM-23, and HZSM-48, HZSM-5. Preferably at least 50% w/w, more preferably at least 90% w/w, still more preferably at least 95% w/w and most preferably 100% of the total amount of molecular sieve used is in the hydrogen form. When the molecular sieves are prepared in the presence of organic cations the molecular sieve may be activated by heating in an inert or oxidative atmosphere to remove organic cations, for example, by heating at a temperature over 500° C. for 1 hour or more. The zeolite is typically obtained in the sodium or potassium form. The hydrogen form can then be obtained by an ion exchange procedure with ammonium salts followed by another heat treatment, for example in an inert or oxidative atmosphere at a temperature over 500° C. for 1 hour or more. The molecular sieves obtained after ion-exchange are also referred to as being in the ammonium form.

The molecular sieve can be used as such or in a formulation, such as in a mixture or combination with a so-called binder material and/or a filler material, and optionally also with an active matrix component. Other components can also be present in the formulation. If one or more molecular sieves are used as such, in particular when no binder, filler, or active matrix material is used, the molecular sieve itself is/are referred to as oxygenate conversion catalyst. In a formulation, the molecular sieve in combination with the other components of the mixture such as binder and/or filler material is/are referred to as oxygenate conversion catalyst. A formulated catalyst can comprise between 1 and 99 wt % aluminosilicate, preferably between 10 and 60 wt %, more preferably between 20 and 50 wt %, based on total catalyst.

It is desirable to provide a catalyst having good mechanical or crush strength, because in an industrial environment the catalyst is often subjected to rough handling, which tends to break down the catalyst into powder-like material. The latter causes problems in the processing. Preferably the molecular sieve is therefore incorporated in a binder material. Examples of suitable materials in a formulation include active and inactive materials and synthetic or naturally occurring zeolites as well as inorganic materials such as clays, silica, alumina, silica-alumina, titania, zirconia and aluminosilicate. For present purposes, inactive materials of a low acidity, such as silica, are preferred because they may prevent unwanted side reactions which may take place in case a more acidic material, such as alumina or silica-alumina is used.

Typically the oxygenate conversion catalyst deactivates in the course of the process. Conventional catalyst regeneration techniques can be employed. The catalyst particles used in the process of the present invention can have any shape known to the skilled person to be suitable for this purpose, for it can be present in the form of spray dried catalyst particles, spheres, tablets, rings, extrudates, etc. Extruded catalysts can be applied in various shapes, such as, cylinders and trilobes. If desired, spent oxygenate conversion catalyst can be regenerated and recycled to the process of the invention. Spray-dried particles allowing use in a fluidized bed or riser reactor system are preferred. Spherical particles are normally obtained by spray drying. Preferably the average particle size is in the range of 1-200 μm, preferably 50-100 μm.

The preferred embodiment of step (b) described hereinabove is preferably performed in an OTO conversion zone comprising a fluidized bed or moving bed, e.g. a fast fluidized bed or a riser reactor system, although in general for an OTO process, in particular for an MTP process, also a fixed bed reactor or a tubular reactor can be used. Serial reactor systems can be employed.

In one embodiment, the OTO conversion zone comprises a plurality of sequential reaction sections. Oxygenate can be added to at least two of the sequential reaction sections.

When multiple reaction zones are employed, an olefinic co-feed is advantageously added to the part of the dimethylether-rich feed that is passed to the first reaction zone.

The preferred molar ratio of oxygenate in the oxygenate feedstock to olefin in the olefinic co-feed provided to the OTO conversion zone depends on the specific oxygenate used and the number of reactive oxygen-bonded alkyl groups therein. Preferably the molar ratio of oxygenate to olefin in the total feed lies in the range of 20:1 to 1:10, more preferably in the range of 18:1 to 1:5 and still more preferably in the range of 15:1 to 1:3.

A diluent can also be fed to the OTO conversion system, mixed with the oxygenate and/or co-feed if present, or separately. A preferred diluents is steam, although other inert diluents can be used as well. In one embodiment, the molar ratio of oxygenate to diluent is between 10:1 and 1:10, preferably between 4:1 and 1:2, most preferably between 3:1 and 1:1, such as or 1.5:1, in particular when the oxygenate is methanol and the diluent is water (steam).

The olefinic co-feed optionally provided together with the oxygenate feedstock to the OTO conversion zone may contain one olefin or a mixture of olefins. Apart from olefins, the olefinic co-feed may contain other hydrocarbon compounds, such as for example paraffinic, alkylaromatic, aromatic compounds or a mixture thereof. Preferably the olefinic co-feed comprises an olefinic fraction of more than 20 wt %, more preferably more than 25 wt %, still more preferably more than 50 wt %, which olefinic fraction consists of olefin(s). The olefinic co-feed can consist essentially of olefin(s).

Any non-olefinic compounds in the olefinic co-feed are preferably paraffinic compounds. If the olefinic co-feed contains any non-olefinic hydrocarbon, these are preferably paraffinic compounds. Such paraffinic compounds are preferably present in an amount in the range from 0 to 80 wt %, more preferably in the range from 0 to 75 wt %, still more preferably in the range from 0 to 50 wt %.

By an unsaturate is understood an organic compound containing at least two carbon atoms connected by a double or triple bond. By an olefin is understood an organic compound containing at least two carbon atoms connected by a double bond. The olefin can be a mono-olefin, having one double bond, or a poly-olefin, having two or more double bonds. Preferably olefins present in an olefinic co-feed are mono-olefins. C4 olefins, also referred to as butenes (1-butene, 2-butene, iso-butene, and/or butadiene), in particular C4 mono-olefins, are preferred components in the olefinic co-feed.

Preferred olefins have in the range from 2 to 12, preferably in the range from 3 to 10, and more preferably in the range from 4 to 8 carbon atoms.

Examples of suitable olefins that may be contained in the olefinic co-feed include ethene, propene, butene (one or more of 1-butene, 2-butene, and/or iso-butene (2-methyl-1-propene)), pentene (one or more of 1-pentene, 2-pentene, 2-methyl-1-butene, 2-methyl-2-butene, 3-methyl-1-butene, and/or cyclopentene), hexene (one or more of 1-hexene, 2-hexene, 3-hexene, 2-methyl-1-pentene, 2-methyl-2-pentene, 3-methyl-1-pentene, 3-methyl-2-pentene, 4-methyl-1-pentene, 4-methyl-2-pentene, 2,3-dimethyl-1-butene, 2,3-dimethyl-2-butene, 3,3-dimethyl-1-butene, methylcyclopentene and/or cyclohexene), heptenes, octenes, nonenes and decenes. The preference for specific olefins in the olefinic co-feed may depend on the purpose of the process, such as preferred production of ethylene or propylene.

In a preferred embodiment the olefinic co-feed preferably contains olefins having 4 or more carbon atoms (i.e. C₄+ olefins), such as butenes, pentenes, hexenes and heptenes. More preferably the olefinic fraction of the olefinic co-feed comprises at least 50 wt % of butenes and/or pentenes, even more preferably at least 50% wt of butenes, and most preferably at least 90 wt % of butenes. The butene may be 1-, 2-, or iso-butene, or a mixture of two or more thereof.

The process according to the present invention can also be described as a process for the preparation of a lower olefin product, which process comprises the steps of

a) cracking a light paraffin feedstock under cracking conditions in a cracking zone to obtain a cracker effluent comprising lower olefins;
b) converting an oxygenate feedstock in an oxygenate-to-olefins conversion system, comprising a reaction zone in which an oxygenate feedstock is contacted with an oxygenate conversion catalyst under oxygenate conversion conditions, to obtain a conversion effluent comprising lower olefins;
c) combining at least part of the cracker effluent and at least part of the conversion effluent to obtain a combined effluent, and separating a lower olefin product stream from the combined effluent,
wherein the cracker effluent and/or the conversion effluent comprises a C4 portion comprising unsaturates, and wherein the process further comprises at least partially hydrogenating at least part of the C4 portion, to obtain an at least partially hydrogenated C4 feedstock; and recycling at least part of the at least partially hydrogenated C4 feedstock as recycle feedstock to step a) and/or step b).

What is claimed is:

1. A process for the preparation of an olefin product comprising ethylene and/or propylene, which process comprises the steps of
a) cracking a paraffin feedstock comprising C2-C5 paraffins under cracking conditions in a cracking zone to obtain a cracker effluent comprising olefins;
b) converting an oxygenate feedstock in an oxygenate-to-olefins conversion system, comprising a reaction zone in which an oxygenate feedstock is contacted with an oxygenate conversion catalyst under oxygenate conversion conditions, to obtain a conversion effluent comprising ethylene and/or propylene;
c) combining at least part of the cracker effluent and at least part of the conversion effluent to obtain a combined effluent, and separating an olefin product stream comprising ethylene and/or propylene from the combined effluent, wherein the cracker effluent and/or the conversion effluent comprises a C4 portion comprising unsaturates, and wherein the process further comprises at least partially hydrogenating at least part of the C4 portion, to obtain an at least partially hydrogenated C4 feedstock; and recycling at least part of the at least partially hydrogenated C4 feedstock as recycle feedstock to step a) and/or step b).

2. A process according to claim 1, wherein the recycle feedstock comprises butene and wherein the recycle feedstock comprising butene is recycled to step b).

3. A process according to claim 2, wherein the recycle feedstock is contacted with the oxygenate conversion catalyst in the oxygenate-to-olefins reaction zone.

4. A process according to claim 2, wherein the oxygenate-to-olefins conversion system comprises an olefin cracking zone separate from the oxygenate-to-olefins reaction zone, and wherein the recycle feedstock is fed to the olefin cracking zone.

5. A process according to claim 1, wherein the C4 portion comprises butadiene, and wherein the recycle feedstock comprises butene obtained from hydrogenating butadiene.

6. A process according to claim 1, wherein the recycle feedstock comprises butane and wherein the recycle feedstock comprising butane is recycled to step a).

7. A process according to claim 6, wherein the cracking conditions in the cracking zone are selected such that 97 wt % or less of the butane is converted during a single pass through the cracking zone.

8. A process according to claim 1, wherein the recycle feedstock comprises butane and wherein the process further comprises cracking the recycle feedstock comprising butane under low severity cracking conditions, wherein butane conversion is 90 wt % or less, to obtain a butane cracking effluent, and feeding part or all of the butane cracking effluent to step b).

9. A process according to claim 8, further comprising feeding steam to the oxygenate-to-olefins conversion system, and wherein the recycle feedstock is added to the steam, before or after subjecting the steam to superheating.

10. A process according to claim 9 wherein the superheating is conducted in a superheating zone in a convection section of a cracking furnace.

11. A process according to claim 6, wherein at least part of the butane is obtained by hydrogenating unsaturates from the C4 portion.

12. A process according to claim 6, wherein the recycle feedstock that is subjected to cracking conditions comprises less than 10 wt % unsaturates.

13. A process according to claim 6, wherein the recycle feedstock that is subjected to cracking conditions comprises butane obtained by first selectively hydrogenating a C4 fraction of the combined effluent to obtain a partly hydrogenated C4 effluent, and further hydrogenating the partly hydrogenated C4 effluent to convert butene to butane.

14. A process according to claim 1, wherein the light paraffin feedstock comprises ethane, and wherein the cracking conditions in the cracking zone are selected such that 60 wt % or less of the ethane in the light paraffin feedstock is converted during a single pass through the cracking zone.

15. An integrated system for the preparation of an olefin product comprising ethylene and/or propylene, which system comprises
a) a steam cracking system having one or more inlets for a paraffin feedstock comprising C2-C5 paraffins and steam, and an outlet for a cracker effluent comprising C2-C4 olefins;
b) an oxygenate-to-olefins conversion system, having one or more inlets for receiving an oxygenate feedstock, and comprising a reaction zone for contacting the oxygenate feedstock with an oxygenate conversion catalyst under oxygenate conversion conditions, and an outlet for a conversion effluent comprising C2-C4 olefins;
c) a work-up section arranged to receive at least part of the cracker effluent and at least part of the conversion effluent to obtain a combined effluent, the work-up section comprising a separation system, an outlet for a lower olefin product stream, and a hydrogenation unit for at least partially hydrogenating at least part of a C4 portion from the combined effluent, an outlet for an at least partially hydrogenated C4 feedstock; and means for recycling at least part of the at least partially hydrogenated C4 feedstock to step the steam cracking system and/or the oxygenate-to-olefins conversion system.
